(19) United States
(12) Patent Application Publication
(10) Pub. No.: US 2003/0105121 A1
(43) Pub. Date: Jun. 5, 2003

(54) Method of preventing lipodystrophy syndrome or reversing a pre-existing syndrome in HIV-infected patients being treated with antiretroviral agents

(76) Inventor: Bernard Bihari, New York, NY (US)

Correspondence Address: WILLIAM J. DANIEL, 6100 WOODLAND TERRACE, MCLEAN, VA, 22101, US

(21) Appl. No.: 10/341,441

(22) Filed: Jan. 14, 2003

Related U.S. Application Data
(63) Continuation-in-part of application No. 09/613,251, filed on Jul. 10, 2000, now abandoned.
(60) Provisional application No. 60/145,843, filed on Jul. 27, 1999.

(51) Int. Cl. A61K 31/485

(52) U.S. Cl. 514/282

(57) ABSTRACT

An improvement in a method of treating an HIV/AIDS infection in human patients in which the patient receives antiretroviral therapy and is consequently subjected to significant risk of developing the lipodystrophy syndrome in one or more of its characteristics. This risk is reduced, and pre-existing signs of such syndrome from past therapy can be substantially reversed, by the concurrent administration by a therapeutically effective mode of an essentially pure opiate receptor antagonist such as Naltrexone and Naloxone at a low level dosage.

### CROSS-REFERENCE TO PRIOR APPLICATION

This application is a continuation-in-part of my prior application Ser. No. 09/613,251, filed Jul. 10, 2000, as a complete application of PROV. S. No. 60/145,843, filed Jul. 27, 1999.

### SPECIFICATION

This invention relates to the treatment of HIV-infected patients who are already undergoing or about to undergo treatment by an antiretroviral therapy employing at least one antiretroviral agent, especially a protease inhibitor and optionally at least one reverse transcriptase inhibitor, selected from either or both of the nucleoside and non-nucleoside categories, and are consequently at significant risk of developing lipodystrophy syndrome or have already developed such syndrome from past therapy, by the concurrent administration with such antiretrovireal agent(s) of an essentially pure opiate receptor antagonist such as Naltrexone and Naloxone by a therapeutically effective mode at a low level dosage which produces substantially the therapeutic results corresponding to those obtained by the administration of Naltrexone at a low dosage level in the range of 1.0 mg. to 10 mg. and preferably 1.0 to 4.5 mg., generally during the night hours, e.g. bedtime.

### BACKGROUND

Since emerging about 1977, acquired immunodeficiency syndrome (AIDS) and its underlying HIV infection has become a major medical problem in the United States and even more so elsewhere in the world. According to “The Merck Manual of Medical Information”, Home Edition, Copyright 1997 by Merck and Co., Inc., through roughly the end of 1995, more than one-half million cases had been reported in the U.S. with about 300,000 deaths attributable to this syndrome. The number of persons infected with its causative virus, human immunodeficiency virus (HIV), is currently believed to be about double the number of active cases. The virus is transmitted via contact with the bodily fluids of an infected persons, particularly with mucous membranes or sera, and its attack was initially concentrated in the homosexual and injecting drug-using population. More recently, however, the rate of infection in heterosexuals is rapidly increasing with women making up 10% of the cases and increasing at a more rapid rate than men. There is, therefore, as compelling need for improved modalities for treating AIDS and/or achieving control over the spread of HIV systemically in a given individual.

I have already disclosed in U.S. Pat. No. 4,888,346, issued Dec. 19, 1989, as a continuation of Ser. No. 06/916,180, filed Oct. 7, 1986, the administration of an essentially pure opiate receptor antagonist at a low dose level to patients suffering from acquired immune deficiency syndrome (or AIDS) in any of its known states, including AIDS-related complex. For this treatment, the amount of the essentially pure opiate receptor antagonist was required to be at a quite low level corresponding in results to those obtained by the administration of Naltrexone at a dosage level of from 1.0 mg. to 10 mg., preferably at a dosage level of 1.0 mg. to about 5 mg., and most preferably up to about 3.0 mg, all per day. At dosage levels above about 10 mg. per day, not only were the desired therapeutic results not obtained but the effect of the treatment appeared to be negative in acerbating the disease.

In as much as naltrexone received some years ago governmental approval for use at a 50 mg. level for treatment of heroin dependence, administration of this drug at the levels specified above is quite safe and has been accompanied by minimal side effects or evidence of toxicity. Long experience has shown the perhaps three percent (3%) of patients receiving the drug at a 4.5 mg. dosage per day may suffer sleep disturbance lasting more than two or three days. Where this has been observed, reduction of the dosage to 3.0 mg. generally returns the sleep pattern to normalcy.

Experience with the low dose naltrexone treatment of HIV showed that while the naltrexone was beneficial in slowing the progression of the infection from whatever state existed at the time to full-blown AIDS, that is, it exerted a stabilizing effect on the patient's condition, it was usually unable to reverse or improve that condition to any substantial degree, e.g. in terms of substantially improving the CD4 blood count, HIV-PCR blood level or other objective measures of HIV infection.

Since that disclosure, a great deal of medical research has been, and still is, being carried out and as a result, the treatment of HIV infections and AIDS has undergone tremendous changes, In particular, this research has resulted in a number of anti-viral drugs (or antiretroviral drugs, the two terms being used synonymously herein) for directly attacking the HIV virus and offering the possibility of actually reversing the extent of HIV infection. The first group of such drugs were nucleoside reverse transcriptase inhibitors or “nucleoside analogues” (NA's) such as AZT (zidovudine), sold under the name “Retrovir, ddI (didanosine), sold under the name “Videx”, ddC (zalcitabine), sold under the name “HIVID”, d4T (stavudine), sold under the name “Zerit”, 3TC (lamivudine), sold under the name “Epivir”, and recently, abacavir, sold under the name “Ziagen”. A combination of lamivudine and a newer NA zidovudine has become available under the name “Combivir”.

Another group is the non-nucleoside reverse transcriptase inhibitors (NNRTI) including nevirapine, sold under the name “VIRAMUNE” efavirenz, sold under the name “BUSTIVA”, and delavirdine. A subsequent and highly potent class, approved by the FDA only in January, 1996, are the protease inhibitors (PI), represented by saquinavir, sold under the names “Inverase” and “Fortovase”, ritonavir, sold under the name “Norvir”, indinavir, sold under the name “Crixivan”, nelfinavir, sold under the name “Viracept”, and lamprenavir, sold under the name “Agenerase”. All of the anti-viral agents thus far mentioned are described in detail in the 2000 and 2002 editions, inter alia, of the Physicians Desk Reference (PDR) published by the Medical Economics Company, Inc. at Montvale, N.J.

Quite recently, a new PI agent has become available under the name “kaletra”. This new agent is a combination of a new PI lopinavir and the known PI ritonavir (norvir) referred to above. Experience with the use of kaletra has shown it to have reduced side effects compared to the previously available PI agents and it therefore has become the PI agent of choice for the treatment of most patients. The inclusion of ritonivir appears to slow the liver metabolism of lopinavir sufficiently to maintain therapeutic blood levels of the latter for 12-18 hours. A detailed description of kaletra can be found in the 2002 edition of PDR but not in prior editions.

It is expected that new PI anti-viral agents are in the course of development and will likely win governmental approval. Because the effects of prolonged PI administration next to be described are so far as can be ascertained inherent in the function of such agents, any new PI agents that become available in the future can be predicted with reasonable certainty to behave in the same manner as those PI agents already in use and are deemed to be within the scope of this invention. This conclusion is confirmed by the action of the latest addition to the PI “repertoire”, kaletra, and its immediate incorporation into the clinical therapy of HIV.

The PI agents are more potent than the reverse transcriptase inhibitors, with the possible exception of “Ziagen”, with generally less serious side effects and their availability for the past two years has literally revolutionized AIDS and HIV therapy. When properly handled, they are often able to convert AIDS/HIV from a uniformly fatal to a chronic manageable disease. The success experienced with the PI agents created great excitement in the field and, in fact, after about the first year of their use raised hopes of an eventual cure for HIV infection.

Unfortunately, there have since been two developments that have seriously tempered the enthusiasm initially aroused by the PI anti-virals: First, even when these anti-viral agents are utilized in combination with others of the same class or of different classes, such as NA and NNRTI, as is routinely recommended for most effective results, the HIV virus after a period of use began to show significant resistance to these drugs and “breaks through” from a low, often undetectable, state into a diagnostically detectable range. This transition in resistance has been the usual experience with the antiviral drugs, occurring after periods of time varying widely according to the drugs selected and the “stamina” of the patient's immune system. Generally, from an initial apparently complete suppression of the blood levels of HIV (viral undetectability) in about 80% of patients, after about 9 to 15 months of use, the virus “broke through” and re-appeared in more than about three-quarters of these patients.

Second, a number of patients began to experience a repulsive physical side effect which is appears to be common to the PI anti-virals as a class and is sometimes found to be associated with other types of anti-viral agents. This side effect is a lipodystrophy syndrome that was first noted in HIV-positive patients being treated with the new PI anti-viral drugs in mid-to-late 1997 and reported extensively at the XI International AIDS Conference in Geneva in June, 1998, This syndrome gradually appears following several months of treatment with PI's and involves changes in fat and sugar metabolism leading to marked elevation in serum cholesterol levels, increases in blood sugar levels culminating in diabetes, combined with redistribution of body fats leading to gross and repulsive changes in body shape. Thus, the arms and legs (extremities) become quite thin, losing a great deal of their fat and musculature, while fat deposits increase (often markedly) in the lower abdomen, breasts, and back of the neck (sometimes referred to as “buffalo hump”, as is encountered in Cushing's syndrome). In studies presented in the 1998 Geneva Conference, this syndrome occurred during the first year of treatment with PI's in varying percentages of patients ranging from a low of 11% to a high of 44%. Further experience has shown that the risk of a patient developing one or more characteristics of lipodystrophy within the first year of treatment with combinations of antiretroviral agents including at least one of the PI group varies between 18% and 50%, depending upon the particular study

While this side effect of lipodystrophy is especially associated with anti-viral agents of the PI class, it has been determined that treatment with the NRTI agent zerit (stavovidine) is also accompanied by a high rate of inducing lipodystrophy (at least 40% for that drug). With combinations of antiviral agents exclusive of a PI agent or zerit, the risk of encountering lipodystrophy is generally 12% to 18%. The only other modalities occasionally used in HIV treatment that are accompanied by a significant increase in the risk of lipodystrophy are the corticosteroids such as prednisone, dexamethasone, prednisolone or solu-medrol. These can sometimes be prescribed to aid in the management of some opportunistic infections or cancers associated with HIV infections.

So far as can be ascertained, the benefits of the present invention of significantly lowering the risk of developing lipodystrophy are applicable independently of the particular therapeutic cause of, or therapeutic factor contributing to, the lipodystrophy. Hence, the present invention extends to treatment of patients with HIV/AIDS by means of antiretroviral agents generally whether or not a PI agent is included in such treatment. For convenience, the term “antiretroviral therapy” is used herein to refer to the treatment of HIV/AIDS in patients by means of antiretroviral agents generally including combinations of such agents as is typically the case.

Given the particularly high rate of lipodystrophy associated with the NA zerit, the choice of this agent is generally contraindicated but, of course, if the patient has ceased to respond to treatment with other antivirals, then resort to zerit may be unavoidable and in that event, the availability of the present invention is a distinct advantage.

### SUMMARY OF THE INVENTION

I have discovered that the administration to a patient having significant blood levels of HIV virus of an essentially pure opiate receptor antagonist at a therapeutically effective low dose level concurrently with the administration in therapeutic amounts of one or more antiretroviral agents, in particular of the PI class either alone or optionally and more usually in combination with at least one NA and/or NNRTI antiviral agent, i.e. an antiretroviral therapy, serves to enhance the anti-viral activity of the antiviral agents by preventing break-through of the virus for prolonged periods, compared to results reported with the antiviral agents alone (i.e. exclusive of the essentially pure opiate receptor antagonist), while significantly reducing for prolonged periods of time the risk presented by such treatment of the patient developing one or more of the signs or characteristics of the lipodystrophy syndrome. Moreover, the possibility has been found to exist of reversing or turning around by the low-dose administration of the pure opiate receptor antagonist a lipodystrophy condition pre-existing in a patient as a consequence of past antiretroviral therapy with anti-viral agents.

### DETAILED DESCRIPTION OF THE INVENTION

One must understand at the beginning that HIV infections and AIDS are not distinct or different diseases; rather, AIDS is the culmination of a progressively exacerbated HIV infection. Indeed, according the “Merck Manual”, supra, the HIV virus begins reproduction immediately following initial entry into the body and very soon reaches a contagious state. The initial symptoms apparent after a few weeks are relatively mild, resembling infectious mononucleosis, viz. fever, rashes, swollen lymph glands, weight loss, diarrhea, and general discomfort, lasting perhaps up to two weeks. These symptoms can be repeated and can go on for years before the AIDS stage is reached. There is, of course, a highly accurate blood test (of the ELISA type) for detecting the presence of antibodies to the HIV virus that is routinely employed in monitoring blood donor programs.

The defining characteristics of full-blown AIDS are actually secondary or indirect in the form of sarcoma, non-Hodgins lymphoma and/or so-called opportunistic infections (OI) that are ordinarily relatively easily thrown off by a person with a normally healthy immune system but become insidious for one with an immune system ravaged by the virus and lead eventually to death. In addition, a CD4 lymphocyte cell count reduced from a normal level of 800 to 1,300 cells per ml of blood to less than 200 cells is a diagnostic indication of progression into full-blown AIDS. Incidentally, the CD4 count is a measure of the condition of the total immune system of the patient and is therefore indicative not only of the degree of the HIV infection but of the patient's susceptibility to onslaught of OI.

It will thus be appreciated that the transition of an HIV infection into full-blown AIDS is not sharp and distinct and in identifying the invention in terms of the treatment of HIV infections, it is not intended to exclude full-blown AIDS in as much as the HIV infection is still the underlying condition in AIDS but in a more virulent and aggressive level.

At this time, it is not possible to provide a technically plausible explanation of the apparently synergistic or cooperative behavior of the essentially pure opiate antagonists with the protease inhibitor or other antiviral agents. At the time of my research on which the -346 patent is based, the NA and PI antiviral drugs were unknown and did not become available until about ten years later. It was assumed from my earlier research that the opiate receptor antagonist had in some indirect manner an immune enhancing action on the natural immune system of the patient, strengthening the ability of the immune system against further invasion of the HIV virus. In contrast, it is generally accepted that the antiviral drugs of either the NI or PI varieties act by preventing the virus from reproducing usually within a lymphocyte, specifically a CD4 lymphocyte, and thereby slowing the rate of growth of the virus. It is not at all apparent how such [these] quite different therapeutic mechanisms might work together.

The administration of the essentially pure opiate receptor antagonist in accordance with the present invention follows the protocol set forth in the -346 patent above. Thus, the amount of the essentially pure opiate receptor antagonist is required to be at a quite low level selected to produce therapeutic results corresponding to those obtained by the administration of Naltrexone at a dosage level of from 1.0 mg. to 10 mg., preferably at a dosage level of 1.0 mg. to about 5 mg., and most preferably up to about 3.0 mg or in some cases, about 4.5 mg. That is to say, when Naltrexone is the selected opiate receptor antagonist, the amount administered is within these ranges. On the other hand, if the opiate receptor antagonist is other than Naltrexone, the amount is adjusted to give the same therapeutic results as are given by Naltrexone within the specified ranges. This may, of course, call for some empirical evaluation but this can be carried out without significant difficulty. It appears to be important that the maximum specified level not be exceeded. At dosage levels above about 10 mg., not only were the desired therapeutic results not obtained but the effect of the treatment appeared to be negative in exacerbating the disease.

If more complete information as to dosage levels which are applicable to the present invention is desired, reference may be had to the same -739 U.S. patent, especially col. 6, line 59-col. 7, line 17, which is incorporated by reference.

As was also true at the time of my prior patents, Naltrexone and Naloxone are presently the only essentially pure opiate receptor drugs known to have received government approval for administration to humans. However, it is certainly conceivable that other drugs of this type might be developed in the future and if this should occur, they should possess the same characteristics as naltrexone and naloxone and would then fall within the scope of the invention.

To avoid any misunderstanding as to the meaning of the modifier “essentially pure antagonist”, this denotes a preferential or selective affinity of the antagonist for Mu over Delta opiate receptor sites, as distinguished from a so-called “mixed antagonist” which has a more or less equal affinity for both groups of receptor sites. It should be borne in mind that the preferential action of the antagonist on a specific groups of opiate receptors is correlated to the dosage at which the antagonist is administered. The dosage levels described above are chosen to take advantage of a selectively higher blocking action of the antagonist against Mu opiate receptors than against Delta receptors by insuring that the amount of the antagonist does not override this preferential action.

Further, by properly limiting the amount of the antagonist within the ranges stated, one can maximize the preferential action and achieve a blocking action that is substantially exclusive for Mu opiate receptors with little or no blocking action exerted against Delta opiate receptors. The narrower range is designed with this virtual exclusivity in mind and is preferred for this reason.

For more details of this selective or preferential action, reference may be had to U.S. Pat. No. 5,013,739, the relevant portions of which, in particular col. 5, line 17-col. 6, line 25, are incorporated by reference.

Similarly, while the dosage levels have been briefly specified above, more complete information as to dosage level which is applicable to the present invention is given in the same -739 U.S. patent, especially col. 6, line 59-col. 7, line 17, which is incorporated by reference. In as much as Naltrexone is available in a form suitable for oral administration and is recognized to be effective when so administered, it is preferred that the Naltrexone be utilized as the opiate antagonist and be administered orally once a day, but where effective other administration routes are, in principle, not precluded and can be employed. Naloxone, on the other hand, has not generally proven to be effective when administered orally; it is available in a form suitable for injection and is better administered by injection, If other essentially pure opiate receptor antagonists should become available, the mode of administration should obviously follow the manufacturer's direction.

It is now known to be important that administration of the essentially pure opiate receptor antagonist take place in the evening hours between about 9 PM and 3AM, e.g. at bedtime (qhs), since the action of the antagonist appears to take place during this time. For brevity, administration of naltrexone in the dosage range and manner set forth above may, from time to time herein, be generally referred to as “LDN”.

It should be understood that the present invention is neither directed to or concerned with the discovery, identification and development of new antiviral agents as such. Such work is the focus of the pharmaceutical industry and requires a great deal of specialized research and skills. Rather, this invention is aimed at the mitigation of problems that have been encountered by practicing physicians involved in the clinical therapeutic treatment of patients with HIV/AIDS infections by means of anti-viral agents that have already been developed by the pharmaceutical industry and have been made reasonably available to practicing physicians for prescription to patients.

In light of the above, the dosage level and mode of administration of the antiviral agent(s) in the antiretroviral therapy should proceed according to the usual practice with such agents and particularly as recommended by the pharmaceutical company by which they are supplied. As with most medications, ranges of the therapeutically effective levels in the blood of the patient have been established for available antiretroviral agents by extensive and complicated trials conducted by the sponsoring company under the supervision of the Food and Drug Administration and, in general, a dosage amount of the anti-viral agent appropriate to achieve a level within the approved range for that agent should be employed. However, adherence to these standard protocols need not be slavish since as will be explained later, some variation from these recognized effective blood levels has been found possible for certain of the antiviral drugs without reducing the effectiveness of the treatment. For example, the treatment of the invention has been found to be effective with a dose of certain antiviral agents identified above that achieves a blood level for the drug significantly below the generally recognized minimum level. This may be influenced by differences in the natural resistance of particular patients and, of course, caution should be exercised in any deviation from the standard blood levels proscribed for the specific agent in question.

Also, for antiretroviral therapy involving certain combinations of antiviral agents from the different groups, adjustment in the amount of one may be required (or at least advisable) to compensate for any negative effect of one drug upon the blood level of the other. For example, for the combination of the PI indinavir with the NNRTI nevirapine, it was found necessary to increase the amount of indinavir from the quantity generally capable of a blood level within the effective range, by 20%, i.e. from 800 mg. every eight hours to 1000 mg. because the nevirapine induced a reduction in the actual blood level of indinavir at the lower dosage amount.

As will be inferred from the above and in contrast to the usual administration of the essentially pure opiate receptor antagonist of a single dose per day, multi-daily dosages may be needed for some or all of the antiviral agents in order to maintain therapeutically effective blood levels therefor. Again, this is in accordance with accepted treatment modalities applicable to the specific drugs. If improved anti-viral drugs are developed that call according to the manufacturer's studies for only a single dose per day, as is currently on the horizon, then naturally the prescribed dosage should be used, absent special considerations.

Also, it may prove desirable based on a given patient's response to the treatment, i.e. CD4 count, HIV-PCR level, etc., to begin a course at one dosage level and either increase, or even decrease, that level as best fits the requirements for that patient. Obviously, this envisions careful monitoring of the blood levels of the antiviral drug(s) being administered and equally or even more important, of the diagnostic indicators of the state of the HIV infection as treatment proceeds and an intelligent response to changes in these factors.

In light of the above considerations, it will be understood that the phrase “administration of substantially a therapeutically effective dosage” of any of the medicaments within the scope of this invention is intended to cover administration of an amount of the drug that is sufficient to achieve a blood level thereof within the recognized range therefor with such variation as is found permissible from actual experience and at a daily frequency appropriate for that drug.

Reference has already been made to the propensity of HIV patients to develop opportunistic infections (OI) which at least up until this invention has been virtually unavoidable for many patients. Such infections have no direct relation to the HIV infection. Thus, if an OI exists or occurs during treatment, it should be treated as is called for by good medical practice for the particular infection until the OT has been resolved.

### CASE EXPERIENCE

A. Reducing Risk of Developing Lipodystrophy.

Introduction.

Because individual examples of treatment histories are of little value in discerning the effectiveness of a given treatment for HIV/AIDS infections, and especially effectiveness in reducing the likelihood of developing a highly variable side effect such as lipodystrophy, due among other things to wide differences in the initial extent of the infection as well as in the response of any given individual, a group of 85 different cases will be evaluated as a group in the following discussion. This number is believed to be a sufficiently large statistical sample to permit a reasonable judgment to be made of the effectiveness of the treatment of this invention. All of the 85 patients were available throughout the study (and continue to be available) for follow-up evaluations.

The constituency of the group os as follows:

By race:

White—64

African American—13

Latino—6

Asian American—2

By gender:

Male—75

Female—10

By age:

22 to 74

Treatment Methodology.

This multi-case study was begun in late 1996 when nevirapine (NNRTI) became available and several months after the new PI antivirals (especially indinavir) became widely available. The medication status of the group at the time of initiation of PI treatment was as follows: Due to historical circumstances, i.e. the predilection in view of the earlier patent of the applicant to treating HIV/AIDS patients with naltrexone, all individuals of the group were already receiving naltrexone at the low dosage level. Based on blood count of CD$+ lymphocytes, individual clinical history and personal patient preference, 60 had already been receiving, or were started on, lamivudine (NA) while 49 of the 65 were also receiving zidovudine (NA) and 4 of the 69 were receiving a combination of lamivudine and stavovudine, bearing in mind that the NA group of antivirals had been available for the longest time. This NA inhibitor treatment had lasted for 3 to 15 months before beginning the inventive treatment, again due to the historical circumstances, i.e. the sequence of entry of the different groups of antiviral drugs into the market. Patients already on any of the NA agents, as just specified, were continued on this treatment during the study. The remaining 25 were receiving no medications (other than naltrexone) at the start of the study.

Patients who at the time were under prophylaxes appropriate for on-going opportunistic infections (OI) were continued on such prophylaxes until the OI had subsided, notwithstanding substantial improvement in CD4+ lymphocyte number and percentage. As noted previously, an HIV infection and OI's associated with an HIV infection have no real pathological connection with one another, other than the HIV infection rendering the individual's system peculiarly vulnerable to attack by of the OI. Therefore, improvement in the state of the HIV infection does not necessarily denote an improvement in the state of the OI; each should be treated separately according to its own protocol.

All patients were started on nevirapine according to its recommended protocol, i.e. 200 mg twice a day, together on the same day with indinavir at a dosage of 1000 mg. every 8 hrs and naltrexone at a dosage of 3 mg. per day taken at bedtime. The indinavir dose was higher than the usual dose of 800 mg. per 8 hrs to compensate for an observed effect of nevirapine in inducing a decreased blood level of indinavir. The mean duration of the indinavir/nevirapine/naltrexone therapy until the latest available laboratory results has been 20 months with more than 50% of the test group lasting 23 months.

Clinical Results.

The results of the study in terms of significant blood levels (analyzed initially after four weeks and thereafter every eight weeks) is as follows:

| | Baseline | Latest Laboratory Results |
|---|---|---|
| HIV RNA-PCR (mean) | 118,383 | <400 for 75 of the group |
| | | (“undetectable”) |
| | | 6,225 for remaining 10 |
| CD4 percentage (mean) | 17.7 | 26.4 |
| CD4 number (mean) | 238 | 448 |

(CD4 percentage is of the total CD4 content in the blood.)

The 75 who achieved “PCR undetectability”, as recorded above, did so within 4 weeks of treatment and have remained at an undetectable state ever since. Six of the remaining 10 were never able to reach undetectibility during this study; three others were able to reach the undetectable level for a single blood test but quickly reverted to a detectible level. All nine of these admitted to significant and prolonged deviations from compliance with the strict indinavir regimen. The final patient who did comply with the regimen did experience sustained suppression of viral load for six months of tests but then showed plasma viremia, i.e. a viral “broke-through”.

All 25 in the sub-group who received only the indinavir/nevirapine/naltrexone combination (i.e. without other anti-virals) reached the undetectable state within one month and have remained in that state since, for durations of 12 to 23 months, with a mean of 20 months. For four of those in the continuous “undetectable” group, during the last six months of the study, indinavir was replaced with a different PI, namely, nelfinavir, for reasons of convenience. Indinavir is required to be taken precisely at eight hour intervals and not followed immediately by ingestion of food in order to maintain the desired constant blood level of the drug, because of the rapid delivery of the drug into the bloodstream, which some patients found burdensome. Nelfinavir, on the other hand, is released into the bloodstream more slowly and need be taken only twice a day on a less exact schedule. This substitution in PI's did not result in any appreciable change in viral levels; however, two of the four switched back to indinavir because of persistent diarrhea associated with the nelfinavir.

It may be of possible interest that of the 75 persistently pcr-undetectable patients, 18 on the occasion of at least two blood analyses had blood levels below the recognized therapeutic values for indinavir of 8.6 to 16.5 mcg/ml, with three of the 18 at a value even below 2.0 mcg/ml, although the quantitative amount of dosage remained the same. In view of these unexpectedly low actual blood levels, the dosage of indinavir was increased during the last nine months of treatment in all of these instances as needed to bring the blood level within the above therapeutic range, which in two cases required as much as 1600 mg every eight hours,

This means that a number of the patients were able to sustain an undetectable viral level for a considerable period despite moderately sub-standard, and in three cases very low, blood levels of indinavir. It would appear to follow that considerable variation in dosages of the anti-viral drugs may be possible without significant diminution in the positive effects of the naltrexone (and thus reasonably within the scope of this invention), although prudence would suggest a conservative approach to such variations.

Side Effects.

As to an occurrence of side effects among the patients, once initial OI's were cleared up, no major OI's developed in any of the 85 participants. One had an episode of shingles; none experienced thrush. Seven of the patients after starting on the protocol of the invention developed within the first or second weeks striking body-wide allergic rashes with hives and considerable itching which subsided slowly over a five to seven day period. Two other patients who are not included in this study since no follow-up testing could be done, likewise experienced such rashes which progressed to include fevers, blisters in the mouth, conjunctivitis and swollen eyelids. In light of this reaction, these two were taken off the treatment less than 16 days after its beginning to prevent development of the Stevens-Johnson syndrome. It is assumed that nevirapine was the cause of these allergic reactions.

Most patients of the group experienced side effects typical of indinavir during the first two to four weeks of treatment but all cleared without any adjustment in treatment dosage. As the present time, after as much as 23 weeks of treatment, the only persistent side effects are a somewhat dry skin in many patients and. for a few, mild nausea one hour after the first morning dose of indinavir.

As to the lipodystrophy syndrome symptoms, only a modest rise in cholesterol levels was observed in many patients which was consistent in most instances with a family history of cardiovascular disease. There were no changes in fat distribution, in serum triglycerides or in blood glucose levels, all indicative of the absence of any occurrence of this syndrome in any member of the group. This is in striking contrast to the results of other reported studies of the effects of administration of PI's which showed a rate of incidence of the syndrome varying from 11 to 35%.

Also on the positive side, all but one patient is presently at ideal body weight or higher and most noted a significant increase in energy, appetite, and mood after involvement in this study. At least one-third of the group reported increased mental clarity with a sharper memory, although it should be noted that complaints of memory impairment had not previously been made by most of these. Six patients had originally experienced significant cognitive impairment as regards memory. This impairment has disappeared completely in four of the six and has improved greatly but not completely in the other two.

Evaluation of Results.

Of the 85 patients involved in this study, 88% experienced continuous HIV viral suppression to an undetectable level for a mean period of 20 months, and a maximum of 23 months combined with an mean elevation in CD4 count of 48%. The nine patients who were never able to achieve an undetectable level nevertheless had an mean CD4 increase of 45% and a decrease in HIV RNA-PCA count from a mean of 123,485 initially to 6,225 without a drop in CD4 level or escalation in viral count during the study, all indicative of valuable improvement in condition. The experience of these nine participants suggests that there is definite merit in continuing the regimen of anti-viral treatment according to the invention even with those patients who are unable at any time to achieve a state of full undetectability or who after reaching the undetectable level for some period undergo a break-through in viral levels.

In several months subsequent to the period covered here, only one patient has undergone a “break-through” in viral load and this patient acknowledged frequent cocaine/alcohol “binges” which caused him to stop compliance with the rigorous schedule which the anti-viral treatment demands for proper results.

In addition, one patient subsequent to this study period has developed lipodystrophy. It transpired that this patient after 15 months of adherence to the inventive treatment without signs of the syndrome, relocated some distance away from the treatment site and was unable to make the necessary trips for regular treatment. At the new location, he was able via other physicians to obtain the indinavir and nevirapine but being unable to find a source for naltrexone, discontinued its use. After six months without naltrexone, he began to undergo change in body shape and some nine months later showed the classic symptoms of lipodystrophy including marked elevation in serum cholesterol and triglyceride levels, mild diabetes, and a gross change in fat distribution that has markedly changed his appearance.

The aforegoing summary was based on the experience of the inventor as of the filing of the original disclosure and as that experience has continued for the above group of patients as well as for new patients, the same favorable results have continued, with no important change in treatment except for the substitution in many cases of the new PI kaletra for the initial PI indinavir. Considering both the original group and those subsequently treated, the number of patients now total well over 150, all remaining free of discernible signs of lipodystrophy. Given the experience of other practitioners active in treating HIV patients as to the significant proportion of patients eventually showing signs of lipodystrophy, as already stated, the possibility of these statistical results occurring randomly is virtually zero.

B. Treatment of Pre-existing Lipodystrophy.

One will have noted that the above case experience was entirely focused on limiting the risk of patients who were at the time free of lipodystrophy symptoms developing such symptoms during their antiretroviral therapy. This is explained by the fact that due to historical circumstances, i.e. the only recent introduction of antiretroviral therapy, the patients in this group were all being treated with naltrexone as described before being exposed to antiretroviral therapy. However, credible evidence has since been collected indicating that the low-dose naltrexone (LDN) protocol of the invention can be effective in reducing the severity of, or even eliminating, already existing symptoms of lipodystrophy resulting from past antiretroviral therapy. This was not anticipated and is considered to be a quite surprising finding. Cases relevant to this finding follow:

### Example a

A 51-year old white male attorney living in Europe had previously been treated with naltrexone according to the “low dose” protocol described herein but in September, 1996, had terminated such treatment when the newer antiretroviral therapies, also described above, became available. Specifically, the antiretroviral treatment he received under advice of a local physician utilized a combination of indinavir (crixivan), retrovir (AZT), epivir (3TC) and neviripine (viramune). Over eight months following initiation of the ARV treatment, he gradually developed a 4″ increase in waist size, as sizable “buffalo hump” on the back of his neck, loss of subcutaneous tissue in his cheeks, and a rise in serum cholesterol levels from 180 to 270. At this point, he contacted the author who recommended that the taking of naltrexone be resumed at a dosage of 3 mg. qhs with no alteration in the ARV program. Over the following nine months, his waist size gradually reduced to its former size, the “buffalo hump” reduced by about 50%, the subcutaneous tissue in his cheeks and forearm returned and his serum cholesterol dropped from 270 to 190. Still concerned by the remaining “buffalo hump”, he attempted unsuccessfully to further reduce its size by rubbing oh testosterone and human growth hormone. Three months after the other symptoms of lipodystrophy had cleared, a personal examination was arranged, which confirmed the above findings, and a modification of the naltrexone protocol was made to increase the dosage from 3.0 to 4.5 mg qhs. Some five weeks later, a telephonic report was made to the effect that the “buffalo hump” had completely disappeared and the condition of this patient has remained stable over the ensuing period.

### Example b

A 42-year white male gym instructor in California suffering from an HIV infection had been stabilized with the LDN treatment but stopped the same on the advice of a local physician who started him on an ARV therapy with indinavir, retrovir and epivir. He remained stable clinically as regards the HIV infection but over the following seven months developed a large hump on the back of his neck, a 6″ increase in waist size and a noticeable increase in neck size, requiring larger size shirt collars. Curious if the stoppage of the LDN treatment was a possible contributing factor to these developments., he was seen and resumption of the naltrexone treatment using 3.0 mg qhs was suggested, as an addition to the ARV program. Over the next ten months, his waist size and neck size both returned to their previous normal state and the “buffalo hump” shrank and eventually disappeared. Since that time, the patient has continued to do well on the combination of the ARV and LDN.

### Example c

A 38-year old white male had been diagnosed with AIDS and was on disability for that reason. He was being seen by several different doctors experienced in the treatment of HIV and had been on the LDN program until the new ARV therapies became available in 1996. It was recommended that he start on the latter using indinavir, retrovir and epivir in conjunction with continued LDN treatment. Without consultation with the present inventor, he soon discontinued using LDN, assuming that the combination of the tow therapies approach was not necessary. Within twelve months, he developed marked loss in subcutaneous tissue in this cheeks, forearms and ankles together with enlarged breasts and a 5” increase in waist size. At this point, he was seen and examination showed that increase in waist size had a quality of marked firmness in contrast with the “rubber tire” shape and soft consistency generally found as otherwise healthy middle age men gain weight and undergo “middle age spread” due to insufficient exercise. He was advised to return to the LDN program at the same dose of 3.0 mg qhs. He did so and all of the signs of lipodystrophy, set forth above, gradually disappeared within an eight-month period.

### Example d

A 58 year-old white Canadian man was undergoing treatment for HIV by means of LDN and was in quite stable condition. He relocated to a more distant part of Canada in 1996 and started being seen by a Canadian doctor specializing in handling HIV cases who prescribed an ARV therapy combining saquinavir, norvir, retrovir and zerit. He also recommended that the LDN program be discontinued which the patient accepted. Within the subsequent twelve months, he developed an 8″ increase in waist size, a significant increase in breast size, a sizable “buffalo hump” and a rise in serum cholesterol from 180 to 360. Furthermore, there was a rise in serum triglycerides from 120 to 620. He was started by the local physician on the anti-cholesterol drug lipitor to try to lower his serum fats and on the suggestion of a friend consulted again with this inventor who proposed renewal of the LDN therapy at a dosage of 3.0 mg qhs in addition to the ARV program. This was done resulting in gradual alteration in the patient's body shape as was evidenced by several Polaroid photographs he provided. After 12 months, his serum cholesterol had dropped to 220 and triglycerides to 140. The increase in waist size partially cleared by about one-half as did the increase in breast size and the “buffalo hump”. While the dual treatment has been continued, there has been no further reduction (or increase) in lipodystrophy symptoms.

### Example e

A 52 year-old white male with HIV, living in Colorado, had been taking LDN until in early 1997 when he started on an ARV therapy with viramune, zerit and epivir as a replacement for his LDN treatment. Over the following ten months, he developed a substantial increase in breast and waist size and a “buffalo hump” with no change in blood lipids. He was seen and examined which led to the recommendation that in addition to the ARV therapy, the LDN program be resumed at a dosage of 3.0 mg qhs. After eight months on the combined programs, he had experienced a complete reversal in the indicated physical signs of lipodystrophy and has since remained stable.

### Example f

A 45 year-old white woman, a recovering alcoholic with an HIV infection was recently presented. After eight months on an ARV therapy using kaletra and combovir, she had developed diabetes (a sometimes complication of lipodystrophy) with marked insulin resistance required administration of 90 units of insulin daily. In addition, her waist size had increased by 8″ and she had acquired a sizable “buffalo hump” on her neck together with increased fat deposits in her breasts. She was then started on LDN at a dosage of 4.5 mg qhs without change in the ARV treatment. After four weeks from being started on the LDN therapy, her insulin need had dropped to 20 units per day and after eight weeks, her “buffalo hump” had largely disappeared and her waist size had decreased by 4″. Continuation of the combined therapy has resulted in a gradual clearing in the residual signs of lipodystrophy.

### Example g

A 42 year-old black male had been treated for an HIV infection for eight months with kaletra and combovir which resulted in an increase in waist size from 34″ to 44″ but no other physical or laboratory indications relevant to lipodystrophy. He was started on LDN at dosage of 4.5 mg qhs and after three months, the waist size had reduced to 40″ without significant exercise or change in diet. He has continued on this combined therapy and apart from a gradual continuing decrease in waist size has remained stable.

A scientifically persuasive explanation of the results achieved in the latter category of cases is speculative at best. It is possible that the LDN protocol of the invention aids in restoring a necessary balance in the human body between endorphins and corticosteroids. It is known that the two families of hormones play a complementary role in the acute stress reaction and in many cellular functions in the body. Lipodystrophy resembles in essential every respect the clinical picture of Cushing's syndrome, which is caused by excessive production of ACTH by the anterior pituitary gland leading to excessively high blood levels of corticosteroids without any balancing increase in levels of endorphins. Similarly, lipodystrophy in patients with HIV/AIDS is associated with elevated blood levels of corticosteroids. In fact, the development of lipodystrophy is accelerated when patients receiving antiretroviral therapy are treated with corticosteroids for some complication of their HIV/AIDS. Studies have shown that serum levels of beta-endorphins are as low as 25% of normal in patients with HIV/AIDS, meaning that the imbalance between the two families of stress hormones has to be considerable and would obviously be aggravated by the addition of treatment with corticosteroids. Increase by the LDN protocol of endorphin levels would tend to correct for this imbalance.

It will no doubt have been noted that in the above study, a combination of two anti-viral drugs, one from the PI and one from the NNRTI classes, was utilized in the therapy, and in some instances where the patient was already taking one or more NA type agents, a larger combination of four of the anti-virals. This regimen was decided upon because the patients in this study were deemed entitled to the most effective medical care for their own survival. Virtually from the start of the HIV epidemic, treatment has been with multiple drugs on the theory (or hope) that different drugs would reinforce the action of one other in preventing the virus from acquiring resistance. This is illustrated by the multiple drugs already being taken by a majority of participating patients before entering into the study. While a PI agent exclusive of other anti-viral agents was not tested here for this reason, it is believed that if under particular circumstances, administration of the essentially pure opiate receptor antagonist in conjunction with a PI drug free of other anti-virals was considered desirable for a given individual, the benefit of the invention would be equally manifest. The lipodystrophy syndrome is, as already indicated, a particularly serious side effect with the PI anti-viral agents (with, up to now, the one exception zerit mentioned above) and is less pronounced for other types of anti-virals but so far as can be judged, the action of naltrexone against this syndrome would be exerted in any case.

Conversely, multiplication of the number of anti-viral agents used for therapy is, as a number of members of the study group indicates, a definite option and one that is certainly within the scope of this invention. Indeed, for patients at a rather advanced state of HIV infection, this would be the approach of choice. Based on present information, which is subject and even likely to change, the preferred additional anti-virals would be lamivudine and stavovudine, both in the NA category.

Apart from the quite new PI agent kaletra, the PI anti-viral agents when administered to patients are subject to a variety of side-effects, the severity of which can differ considerably from patient to patient and from agent to agent for a given patient. The choice of a particular PI agent or agents or combination with one or more PI agents of other anti-virals will necessarily then have to be made under the careful guidance of the attending physician on an “ad hoc” basis and may well have to be modified from time to time as the treatment progresses according to the response of the patient and in all likelihood as new and hopefully improved anti-viral agents are made available to the art.

In the course of this disclosure, reference has been made to a variety of modifications or alterations in the practice of this invention and others will undoubtedly occur to one skilled in the handling of patients with HIV infections, if not to medical practitioners generally. Consequently, it is not intended that the invention be restricted to the specifics of the disclosure but rather broadly interpreted to encompass such variations and alternatives.

Having described my invention, that which is claimed is:

1. A method of reducing the risk of the development of the lipodystrophy syndrome in a human patient who is already being or is about to be treated for an AIDS/HIV infection with substantially a therapeutically effective dosage level of a at least one anti-viral agent subjecting said patient to a significant risk of developing said syndrome, which comprises the step of administering to the patient concurrently with treatment with such anti-viral agent by a pharmacologically effective mode an essentially pure opiate receptor antagonist at a low level dosage which produces substantially the therapeutic results corresponding to those obtained by the administration of Naltrexone at a low dosage level in the range of 1.0 mg. to 10 mg.

2. The method of claim 1 wherein at least one of said anti-viral agents is a protease inhibitor anti-viral agent.

3. The method of claim 1 further comprising the concurrent administration to the patient of a therapeutically effective amount of at least one other anti-viral agent.

4. The method of claim 3 wherein said other anti-viral agent is selected from the non-nucleoside reverse transcription inhibitor.

5. The method of claim 4 wherein a combination of anti-viral agents comprising a protease inhibitor, a non-nucleoside reverse transcription inhibitor. and at least one nucleoside reverse transcription inhibitor are concurrently administered to said patient together with said essentially pure opiate receptor antagonist.

6. The method of claim 1 wherein said antagonist is selected from among Naltrexone and Naloxone.

7. The method of claim 1 wherein said antagonist is Naltrexone.

8. The method of claim 1 wherein said antagonist is administered a low level dosage which produces substantially the therapeutic results corresponding to those obtained by the administration of Naltrexone at a low dosage level in the range of 1.0 mg. to 3 mg.

9. In a method of treatment an HIV/AIDS infection in human patients by an anti-viral therapy subjecting said patient to a significant risk of developing over time a lipodystrophy syndrome, the improvement comprising the administration to the patient concurrently with said anti-viral therapy by a pharmacologically effective mode of an essentially pure opiate receptor antagonist at a low level dosage which produces substantially the therapeutic results corresponding to those obtained by the administration of Naltrexone at a low dosage level in the range of 1.0 mg. to 10 mg.

10. In a method of treating a patient who has been undergoing treatment of an HIV/AIDS infection utilizing an antiviral therapy and has developed as a result of such therapy significant characteristics of a lipodystrophy syndrome, the improvement comprising the administration to the patient concurrently with said anti-viral therapy by a pharmacologically effective mode of an essentially pure opiate receptor antagonist at a low level dosage which produces substantially the therapeutic results corresponding to those obtained by the administration of Naltrexone at a low dosage level in the range of 1.0 mg. to 10 mg.
